(19) United States
(12) Patent Application Publication
(10) Pub. No.: US 2014/0235940 A1
(43) Pub. Date: Aug. 21, 2014

(54) S-shaped Visible Hard Intubation Core

(71) Applicants: Weidong Wang, Taizhou (CN); Fushang Xue, Taizhou (CN); Aihua Chen, Taizhou (CN); Benquan Yang, Taizhou (CN)

(72) Inventors: Weidong Wang, Taizhou (CN); Fushang Xue, Taizhou (CN); Aihua Chen, Taizhou (CN); Benquan Yang, Taizhou (CN)

(73) Assignee: Zhejiang Youyi Medical Apparatus Co., Ltd., Taizhou, Zhejiang (CN)

(21) Appl. No.: 14/347,242

(22) PCT Filed: Sep. 13, 2012
(86) PCT No.: PCT/CN2012/081331

§ 371 Date: Mar. 26, 2014

(51) Int. Cl. A61M 16/04; A61B 1/267; A61B 1/06; A61B 1/00; A61B 1/05

(52) U.S. Cl. CPC A61M 16/0488; A61B 1/00052; A61B 1/05; A61B 1/0676; A61B 1/2673; A61B 1/00048
USPC 600/103

(57) ABSTRACT

A S-shaped visible hard intubating stylet comprises a LCD display, a handler and a hard stylet which are connected successively; the hard stylet has an outer end provided with a camera and a light emitter which are connecting to the LCD display, respectively; the hard stylet is formed to comprise, from one end to the other end, successively, a first linear section, an arc-shaped section, a circular arc-shaped section and a second linear section; a tangential direction of a tail end of the circular arc-shaped section that is adjacent to the arc-shaped section, and an axis direction of the handler, have an included angle which is ranging from no 20°; the circular arc-shaped section has a circular arc radius which is ranging from 45 mm to 75 mm; the circular arc-shaped section has a circular arc corresponding to a central angle which is ranging from 60° to 80°; the second linear section and the axis direction of the handler have an included angle which is ranging from 50° to 70°.

### FIELD OF THE INVENTION

The present invention relates to technical field of medical apparatus and instruments, particularly, to a visible hard intubating stylet used for guiding an endotracheal intubation operation on patients.

### BACKGROUND OF THE INVENTION

As a necessary instrument for endotracheal intubation operation performed in anesthesia department, emergency department and intensive care department of hospital, the laryngoscope has been widely applied in clinical practice. Currently, a laryngoscope with video function usually is guiding a tracheal catheter with intubating stylet to be directly inserted into patient's trachea under video image provided by a camera, or guiding the tracheal catheter to be inserted into patient's tracheal along a guiding slot arranged at one side of laryngoscopic lens under the video image. For example, CN 201328799Y published by SIPO on Oct. 21, 2009 with the title of “electric video anesthetic laryngopharyngoscope used for difficult intubation” described an electric video anesthetic laryngopharyngoscope used for difficult intubation, comprising a laryngoscopic lens with handler, a handler ferrule and a LCD display; the handler ferrule is flexibly connected with the LCD display through a damping rotary-joint, and the laryngoscopic lens with handler is connected with the LCD display in such a manner that the laryngoscopic lens with handler is connected to the handler ferrule through a sleeved snap joint or is connected to a LCD display charger through a handler adaptor equipped with cable, wherein the LCD display is connected to a charging station; the connection between the laryngoscopic lens with hanlder and the handler ferrule through the sleeved snap joint is detachable, and the two are electrically conducted through conductive contacts after snap-connected; a lighting, a camera and a heating-defogging circuit are disposed at the front end of the laryngoscopic lens with handler. In order to clearly see the position of glottis, this laryngoscope is provided with a camera inside the laryngoscopic lens, with a lighting device and a heating circuit disposed at a side of the camera, and with a guiding slot disposed alongside of the laryngoscopic lens. For providing the tracheal catheter with necessary moving space during inserting, the guiding slot shall have a cavity larger than that of the tracheal catheter. In this way, the guiding slot, together with the adjacent laryngoscopic lens, have a relatively wider sectional area, which would not cause any obvious problem during the application on normal patient; however, in case of patient who has difficulty in widely opening his/her mouth or has sever laryngopharyngeal disease or narrow throat, it can't get used effectively at all.

In order to solve such problem, a flexible light-guide fibre or video bronchoscope appears accordingly. They are formed by directly disposing a light-guide fibre or a camera on a soft, flexible catheter. During endotracheal intubation, the tracheal catheter is sleeved onto the soft flexible catheter, so as to be applicable for patient who has difficulty in widely opening his/her mouth or has sever laryngopharyngeal disease or narrow throat. However, since the flexible light-guide fibre or the video bronchoscope has a tube body made from flexible material, it's weak in operability, thus involves risk of circumferential distortion or arc-shaped deformation, or even radial flattening during endotracheal intubation, especially for special patients, which all go against successfully finishing the endotracheal intubation operation.

In order to solve the problem that the flexible light-guide fibre or the video bronchoscope is weak in operability, relevant shaping instruments are developed on after another. The shaping instrument can be sleeved onto the outside of the flexible light-guide fibre or the video bronchoscope, so that the clinical application of the flexible light-guide fibre or the video bronchoscope can be improved by means of the stiffness of the shaping instrument made from metallic material; in spite of this, the surgeon may still have difficulty in precisely manipulate the image capturing orientation of the micro-camera disposed at the front end of the flexible light-guide fibre or the video bronchoscope during operation due to insufficient stiffness of the shaping instrument, thereby increasing labor intensity and reducing the working efficiency of the surgeons, and meanwhile extending the suffering time of the patients. In addition, it requires repeated practices to master the associated technology, and also certain times of operations to maintain the operational skills. Therefore there are increasingly increased demands for clinicians to overcome such difficulties.

### SUMMARY OF THE INVENTION

The present invention mainly solves the technical problem that the existing light-guide fibre or video bronchoscope results in difficulty for a clinician to precisely manipulate the image capturing orientation of the micro-camera due to insufficient stiffness of light-guide fibre or video bronchoscope, by providing a S-shaped visible hard intubating stylet which is convenient for operation, capable of precisely manipulating the image capturing orientation of the camera, improving the working efficiency of the surgeon and reducing the discomfort index of the patient during treatment.

The present invention also solves the problem that an appearance of image color distortion may be resulted by optical properties such as birefringence and double-optical axis when an appropriate visual range is beyond, by providing a S-shaped visible hard intubating stylet in which a LCD display is positioned such that the surgeon's viewing angle under normal operation status is exactly within a favorable observing angle range thereof, so as to obtain clear and accurate images for quickly finishing the endotracheal intubation operation or diagnosis treatment.

The technical problems existed in the prior art are solved by the technical solution of a S-shaped visible hard intubating stylet comprising a LCD display, a handler and a hollow hard stylet; the handler has one end connecting to the LCD display and the other end connecting to one end of the hard stylet; the other end of the hard stylet is provided with a camera and a light emitter therein, which are connecting to the LCD display, respectively; the hard stylet is formed to comprise, from one end to the other end, successively, a first linear section, an arc-shaped section, a circular arc-shaped section and a second linear section; the arc-shaped section along with the circular arc-shaped section exhibit a shape of “S”; a tangential direction of a tail end of the circular arc-shaped section that is adjacent to the arc-shaped section, and an axis direction of the handler, have an included angle which is ranging from no 20°; the circular arc-shaped section has a circular arc radius which is ranging from 45 mm to 75 mm; the circular arc-shaped section has a circular arc corresponding to a central angle which is ranging from 60° to 80°; and, the second linear section and the axis direction of the handler have an included angle which is ranging from 50° to 70°.

The upper respiratory tract of human body, usually, is extending like an arc, and an included angel between the root of tongue and the glottis, typically, is ranging from about 70° to about 90°. However, for some special patients with difficult and complicated diseases, due to abnormal conditions such as throat swelling, the upper respiratory tract thereof, mostly, becomes to extend like or approximate a circular arc with a circular arc radius which is ranging from 45 mm to 75 mm, wherein the circular arc corresponds to a central angle which is ranging from 60° to 80°. For this reason, the circular arc-shaped section is arranged to have a circular arc radius ranged from 45 mm to 70 mm, and have a circular arc corresponding to a central angle ranged from 60° to 80°; the second linear section and the axis direction of the handler have an included angle which is arranged to be ranged from 50° to 70°. In this way, the circular arc-shaped section can be matched with the physiologic curvature of the patent's upper respiratory tract in an even better fashion, so as to facilitate the endotracheal intubation operation as far as possibly. Since the patient with difficult and complicated diseases, always, is lying on the back during emergency treatment, when the visible hard intubating stylet is inserted into the patient's throat, the LCD display connecting to the intubating stylet may deviate from the optimal viewing range of the surgeon who is performing the operation, even if the circular arc-shaped section of the hard intubating stylet is matched with the curvature of the patent's upper respiratory tract. In order to solve such problem, the present hard intubating stylet is further provided with additional arc-shaped section between the circular arc-shaped section and the LCD display, the arc-shaped section is curved in a direction opposite to that of the circular arc-shaped section, so that the hard intubating stylet exhibits a shape of “S” which adjusts the position of the LCD display by 2° to 20° and locates it within the optimal viewing range of the surgeon, thereby the surgeon can clearly observe the image of the patient's upper respiratory tract for quick and accurate determination. At the same time, due to the arrangement of arc-shaped section, it can serve as a handler for the surgeon to rotate the hard intubating stylet to facilitate the operation, so that the surgeon can quickly and precisely manipulate the image capturing orientation of the camera under the guide of the display image and find the glottis to finish the endotracheal intubation operation.

The arc-shaped section may have an arc shape which exhibits an elliptic arc, a circular arc, or other shapes of arc; preferably, the arc-shaped section an arc shape which exhibits a circular arc. The circular arc is easily to be processed, thus reducing the manufacture cost.

The included angle between the tangential direction of the tail end of the circular arc-shaped section that is adjacent to the arc-shaped section and the axis direction of the handler is 10°. According to the heights of most surgeons as well as their standing postures during operation, when the included angle between the tangential direction of the tail end of the circular arc-shaped section that is adjacent to the arc-shaped section and the axis direction of the handler is 10°, the LCD screen of the display is exactly located at the optimal viewing position of the surgeon.

The circular arc-shaped section has a circular arc radius which is 57 mm. The upper respiratory tract of most of special patients with difficult and complicated diseases has a circular arc with a characteristic radius of around 57 mm.

The circular arc-shaped section has a circular arc corresponding to a central angle which is 70° . The upper respiratory tract of most of special patients with difficult and complicated diseases has a circular arc with a characteristic central angle of around 70°.

The light emitter and the camera may be integrally connected or may be separate from one another. Preferably, the light emitter is integrally connected with the camera and located at a side of a viewing aperture of the camera. The inside diameter of the tracheal catheter is defined as 6 mm by industry criterion. In order to be in conformity with such criterion, the light emitter is disposed at a side of a viewing aperture of the camera, and integrally formed with the camera.

A tracheal catheter locator is sleeved onto the outside of the first linear section of the hard stylet. In this way, the S-shaped visible hard intubating stylet can be used to accurately position the tracheal catheter, which allows the surgeon operating with single hand and adjusting the intubating orientation of tracheal catheter with the other free hand.

The present is advantageous in that:

i. since the hard intubating stylet of the present invention that is sleeved into a tracheal catheter is inserted into the patient's upper respiratory tract in a manner such that the inserted portion has a shape matched with the curvature of the upper respiratory tract, it can facilitate the endotracheal intubation operation as far as possible;

ii. since the present invention designs the micro-camera and the light emitter to be disposed at the front end of the hard intubating stylet, it can provide superior precision for the manipulation of the camera orientation;

iii. since the hard intubating stylet of the present invention is further provided with an arc-shaped curvature between the circular arc-shaped section and the LCD display, the position of the LCD display is adjusted by 2° to 20° and thus located within the optimal viewing range of the surgeon, which makes the surgeon capable of clearly observing the image of the patient's upper respiratory tract for quick and accurate determination simply with natural standing posture; this can reduce the labor intensity of the surgeon and improve the success rate of endotracheal intubation operation;

iv. since the hard intubating stylet of the present invention is further provided with an arc-shaped section, curved in a direction opposite to that of the circular arc-shaped section, between the circular arc-shaped section and the LCD display, the hard intubating stylet exhibits a shape of “S”, which makes the surgeon capable of using this arc-shaped section as a handler for rotating the hard intubating stylet and hence to facilitate the manipulation thereof, so that the surgeon can quickly and precisely manipulate the image capturing orientation of the camera under the guide of the screening image and find the glottis to finish the endotracheal intubation operation; this shortens the operation time and eases the pain suffered by the patient;

v. since a dedicated tracheal catheter locator is sleeved onto the outside of the first linear section of the hard intubating stylet, which provides convenience for the surgeon to perform intubation with single hand while adjusting the intubating orientation of tracheal catheter with the other free hand.

### BRIEF DESCRIPTION OF THE DRAWINGS

FIG. 1 is a front structural schematic diagram of the present invention;

FIG. 2 is an enlarged view of part R in FIG. 1;

FIG. 3 is an A-directional schematic diagram of FIG. 1.

Wherein, 1: LCD display; 2: handler; 3: tracheal catheter locator; 4: hard stylet; 41: first linear section; 42: arc-shaped section; 43: circular arc-shaped section; 44: second linear section; 5: micro-camera; 51: LED light; 52: viewing aperture.

### DETAIL DESCRIPTION OF THE EMBODIMENTS

Hereinafter the technical solution of the present invention will be described in details through preferred embodiments in conjunction with drawings appended the description.

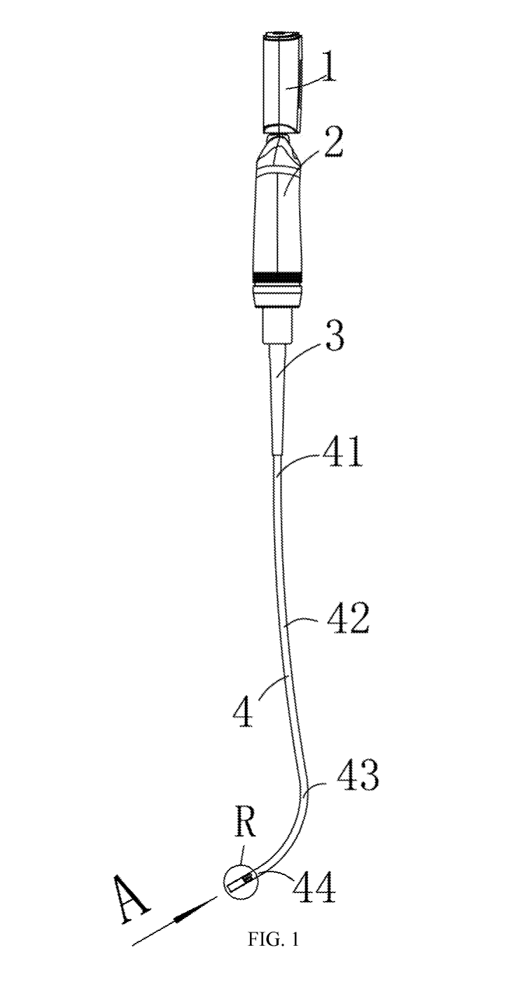

Embodiment: a S-shaped visible hard intubating stylet as shown in FIG. 1 comprises a LCD display 1, a hollow handler 2, a hollow tracheal catheter locator 3, and a hollow hard stylet 4; the handler 2 has one end fixedly connecting to the LCD display 1 and the other end connecting to one end of the tracheal catheter locator 3 through thread; the other end of the tracheal catheter locator 3 is sleeved at one end of the hard stylet 4 with interference fit; the other end of the hard stylet 4 is provided with a micro-camera 5 and a LED light 51 therein, which are connecting to the LCD display, respectively; the hard stylet 4 is formed to comprise, from one end to the other end, successively, a first linear section 41, an arc-shaped section 42, a circular arc-shaped section 43 and a second linear section 44; the arc-shaped section 42 along with the circular arc-shaped section 43 exhibit a shape of “S”; a tangential direction of a tail end of the circular arc-shaped 43 section that is adjacent to the arc-shaped section 42, and an axis direction of the handler 2, have an included angle of 10°; the circular arc-shaped section 43 has a circular arc radius of 57 mm; the circular arc-shaped section 43 has a circular arc corresponding to a central angle of 70°; the second linear section 44 and the axis direction of the handler 2 have an included angle of 60°; For convenience of manufacture, the arc-shaped section 42 has an arc shape which also exhibits a circular arc.

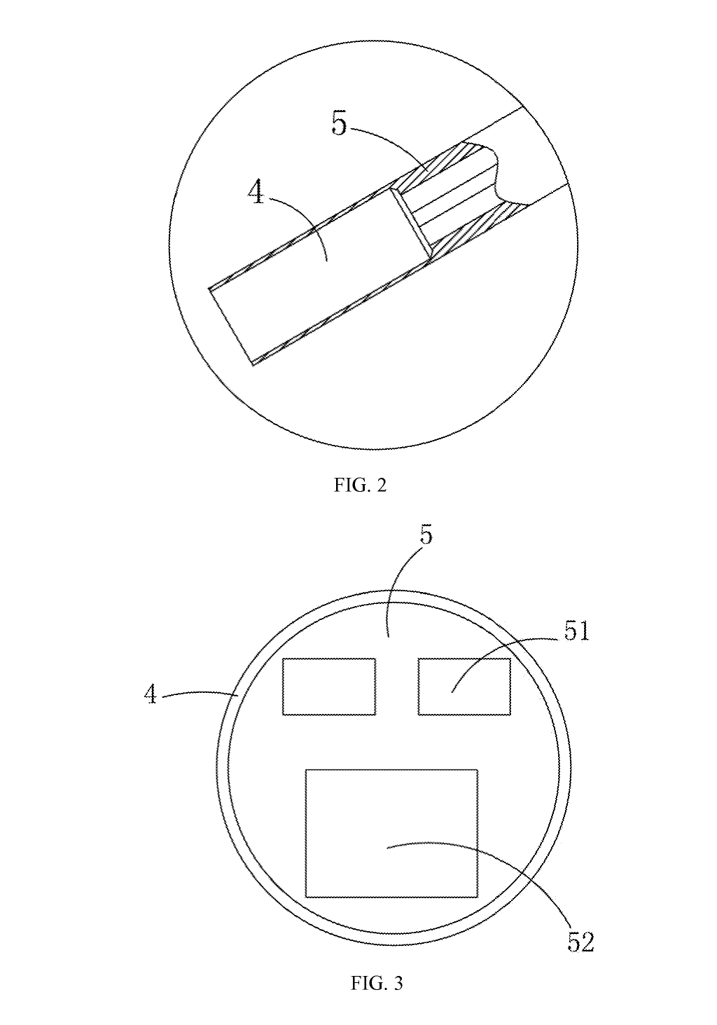

As shown in FIG. 2, the other end of the hard stylet 4 is provided with a mounting cavity, in which the micro-camera 5 is mounted with interference fit; the front end face of the micro-camera 5 is aligned with that of the hard stylet 4.

As shown in FIG. 3, in order to prevent from any optical interference and to obtain high-definition images from LCD display 1, a viewing aperture 52 of the micro-camera 5 is offset relative to an axial center of the micro-camera 5; one side of the viewing aperture 52 of the micro-camera 5 is provided with two LED lights 51, which, along with the viewing aperture 52, are located at two sides of the axial center of the micro-camera 5.

The embodiment described as above is just one of the preferable solutions of the present invention and does not intend to constitute any limitation in any form. Variations and modifications thereof may be made without departing from the technical solutions described in the claims.

The contents that are pertaining to existing technology known to those skilled in the art are not particularly described in the present description.

What is claimed is:

1. A S-shaped visible hard intubating stylet, comprising a LCD display, a handler and a hollow hard stylet; the handler has one end connecting to the LCD display and the other end connecting to one end of the hard stylet;
characterized by that, the other end of the hard stylet is provided with a camera and a light emitter therein, which are connecting to the LCD display, respectively; the hard stylet is formed to comprise, from one end to the other end, successively, a first linear section, an arc-shaped section, a circular arc-shaped section and a second linear section; the arc-shaped section along with the circular arc-shaped section exhibit a shape of “S”; a tangential direction of a tail end of the circular arc-shaped section that is adjacent to the arc-shaped section, and an axis direction of the handler, have an included angle which is ranging from 2° to 20°; the circular arc-shaped section has a circular arc radius which is ranging from 45 mm to 75 mm; the circular arc-shaped section has a circular arc corresponding to a central angle which is ranging from 60° to 80°; the second linear section and the axis direction of the handler have an included angle which is ranging from 50° to 70°.

2. The S-shaped visible hard intubating stylet according to claim 1, characterized by that said arc-shaped section has an arc shape which exhibits a circular arc.

3. The S-shaped visible hard intubating stylet according to claim 2, characterized by that the tangential direction of the tail end of the circular arc-shaped section that is adjacent to the arc-shaped section, and the axis direction of the handler, have an included angle of 10°.

4. The S-shaped visible hard intubating stylet according to claim 1, characterized by that said circular arc-shaped section has a circular arc raidus of 57 mm.

5. The S-shaped visible hard intubating stylet according to claim 4, characterized by that said circular arc-shaped section has a circular arc corresponding to a central angle of 70°.

6. The S-shaped visible hard intubating stylet according to claims 1, characterized by that said light emitter is integrally connected with the camera, and said light emitter is located at one side of a viewing aperture of the camera.

7. The S-shaped visible hard intubating stylet according to claim 1, characterized by that a tracheal catheter locator is sleeved onto the outside of said first linear section of said hard stylet.
